(19) United States
(12) Patent Application Publication
(10) Pub. No.: US 2021/0052143 A1
(43) Pub. Date: Feb. 25, 2021

(54) Control Method, Control Device and Storage Medium for Magnetic Capsule Endoscope Control System

(71) Applicant: Ankon Medical Technologies(Shanghai) Co., LTD., Shanghai (CN)

(72) Inventors: Xiaodong DUAN, Pleasanton, CA (US); Tingqi WANG, WUXI (CN); Xin LIU, Wuxi (CN)

(73) Assignee: Ankon Medical Technologies(Shanghai) Co., LTD., Shanghai (CN)

(21) Appl. No.: 16/988,693

(22) Filed: Aug. 9, 2020

(30) Foreign Application Priority Data
Aug. 9, 2019 (CN) 201910736621.9

(51) Int. Cl. A61B 1/00; H04N 7/18; H04N 5/232; A61B 1/04; A61B 1/045

(52) U.S. Cl. CPC A61B 1/00158; H04N 7/183; H04N 5/23203; H04N 5/23216; A61B 1/041; H04N 2005/2255; A61B 1/00006; A61B 1/0005; A61B 1/00039; A61B 1/00016; A61B 1/045

(57) ABSTRACT

A control method, a control device and a storage medium for a magnetic capsule endoscope control system is provided. The control method includes: displaying a plurality of control buttons in a first area of a first display screen connected to a local industrial personal computer, wherein each control button issues a corresponding control instruction to instruct a target object to execute a specified operation, and the target object is a drive mechanism, a magnetic capsule endoscope or a camera of a control device; receiving a selection operation on any of the control buttons and controlling the corresponding target object to execute the specified operation; displaying images captured by a camera of the magnetic capsule endoscope in a second area of the first display screen.

### CROSS-REFERENCE OF RELATED APPLICATIONS

The application claims priority to Chinese Patent Application No. 201910736621.9 filed on Aug. 9, 2019, the contents of which are incorporated by reference herein.

### FIELD OF INVENTION

The present invention relates to a medical device and more particularly to a control method, a control device, and a storage medium for a magnetic capsule endoscope control system.

### BACKGROUND

Capsule endoscopy has been used more and more clinically because of its features such as no intubation, good comfort, and no cross-infection. The capsule endoscopy examines digestive tract in detail, and the capsule endoscopy is controlled via an external control device.

However, in the process of clinical application and promotion of the capsule endoscope and the external control device, it often encounters problems such as difficulty in operation training and slow mastering of operations, and skilled operation skills require more experience to achieve. Without professional operators, it is difficult to apply the capsule endoscope and the external control device, especially in the areas and emergency rooms short of doctors.

No effective solution has been found for the above problems.

### SUMMARY OF THE INVENTION

The present invention provides a control method, a control device, and a storage medium for a magnetic capsule endoscope control system to at least solve the problem of complex control method of the capsule endoscope control system.

In an embodiment, a control method for a magnetic capsule endoscope control system is provided. The control method is applied to the magnetic capsule endoscope control system, and the magnetic capsule endoscope control system comprises a controlled device, a local industrial personal computer and a magnetic capsule endoscope. The local industrial personal computer controls the controlled device, and the controlled device comprises a magnetic moving part and a drive mechanism for controlling the movement of the magnetic moving part. The control method comprises: displaying a plurality of control buttons in a first area of a first display screen connected to the local industrial personal computer, wherein each of the control buttons issues a corresponding control instruction to instruct a target object to execute a specified operation, the target object is the drive mechanism, the magnetic capsule endoscope or a camera of a control device; receiving a selection operation on any of the control buttons and controlling the corresponding target object to execute the specified operation; and displaying images captured by a camera of the magnetic capsule endoscope in a second area of the first display screen.

Further, the method further comprises: displaying a schematic diagram of the current status of the drive mechanism in a third area of the first display screen; and/or, displaying a schematic diagram of the current status of the magnetic capsule endoscope in a fourth area of the first display screen.

Further, the control system further comprises a remote control equipment connected to the local industrial personal computer through a telecommunication connection to control the controlled device. The local industrial personal computer is installed with a server, the remote control equipment is installed with a client corresponding to the server, and the controlled device comprises a camera disposed towards an examination table of the controlled device. The method further comprises: establishing a telecommunication connection between the local industrial personal computer and the remote control equipment; displaying a plurality of control buttons in a first area of a second display screen connected to the remote control equipment; and displaying images captured by the camera of the controlled device in a second area of the second display screen.

Further, the control system further comprises a remote control equipment connected to the local industrial personal computer through a telecommunication connection to control the controlled device and a console connected to the remote control equipment, where the local industrial personal computer is installed with a server, the remote control equipment is installed with a client corresponding to the server, and the controlled device comprises a camera disposed towards an examination table of the controlled device. The control method further comprises: establishing a telecommunication connection between the local industrial personal computer and the remote control equipment, and a connection between the remote control equipment and the console; displaying a plurality of control buttons on a touch screen of the console; displaying images captured by a camera of the controlled device in a first area of a third display screen connected to the remote control equipment.

Further, the method also comprises: displaying a schematic diagram of the current status of the drive mechanism in a third area of the second display screen; and/or displaying a schematic diagram of the current status of the magnetic capsule endoscope in a fourth area of the second display screen.

Further, the method also comprises: displaying the images captured by the camera of the magnetic capsule endoscope in a fifth area of the second display screen.

Further, the plurality of control buttons comprise custom buttons, the method further comprises: receiving editing operations to the custom buttons; displaying a macro command editing interface, wherein the macro command editing interface comprises one or more of the control buttons and a plurality of input boxes, the input boxes receives parameter information of an input target position, and the parameter information comprises coordinate points and deflection angles; displaying a command currently edited in the macro command editing interface according to the control button selected and/or the parameter information of the target position input in the macro command editing interface; after determining to generate a macro command, generating a macro command from the command currently edited, and assigning the macro command to the custom button, wherein the custom button executes the corresponding macro command after being selected.

In another embodiment, a control device for a magnetic capsule endoscope control system is provided. The control device is applied to the magnetic capsule endoscope control system, and the magnetic capsule endoscope control system comprises a controlled device, a local industrial personal computer and a magnetic capsule endoscope, where the local industrial personal computer controls the controlled device, and the controlled device comprises a magnetic moving part and a drive mechanism for controlling the movement of the magnetic moving part. The control device comprises: a first display module, for displaying a plurality of control buttons in a first area of a first display screen connected to the local industrial personal computer, wherein each of the control buttons issues a corresponding control instruction to instruct a target object to execute a specified operation, the target object is the drive mechanism, the magnetic capsule endoscope or a camera of the control device; an execution module, for receiving a selection operation on any of the control buttons and controlling the corresponding target object to execute the specified operation; and a second display module, for displaying images captured by a camera of the magnetic capsule endoscope in a second area of the first display screen.

Further, the control device also comprises: a third display module, for displaying a schematic diagram of the current status of the drive mechanism in the third area of the first display screen; and/or a fourth display module, for displaying a schematic diagram of the current status of the magnetic capsule endoscope in the fourth area of the first display screen.

In still another embodiment, a storage medium is also provided. The storage medium stores computer programs and the computer programs are set to execute the steps in any of the above embodiments.

In still another embodiment, an electronic device is also provided comprising a memory and a processor, where the memory stores computer programs and the processor is set to run the computer programs to execute the steps in any of the above methods.

The present invention can display a plurality of control buttons in the first area of the first display screen connected to the local industrial personal computer, and each control button issues a corresponding control instruction to instruct a target object to execute a specified operation, and the target object is the drive mechanism, the magnetic capsule endoscope or the camera of the control device. The selection operation on any of the control buttons is received and the corresponding target object is controlled to execute the specified operation. Images captured by the camera of the magnetic capsule endoscope are displayed in the second area of the first display screen. The method solves the technical problem of complex control method for the magnetic capsule endoscope control system.

### BRIEF DESCRIPTION OF THE DRAWINGS

The accompanying drawings here provide a further understanding of the present invention and are incorporated in and constitute a part of the present invention. The schematic embodiments of the present invention and their descriptions are used to explain the invention and do not constitute undue limitations on the present invention.

FIG. 1 shows a flowchart of a control method for a magnetic capsule endoscope control system according to an embodiment of the present invention.

FIG. 2 shows a schematic diagram of an optional display interface of the control method for the magnetic capsule endoscope control system according to an embodiment of the present invention.

FIG. 3 shows a functional schematic diagram of the control method for the magnetic capsule endoscope control system according to an embodiment of the present invention.

FIG. 4 shows a schematic diagram of an optional macro command editing interface of the control method for the magnetic capsule endoscope control system according to an embodiment of the present invention.

FIG. 5 shows a schematic diagram of a control device of the magnetic capsule endoscope control system according to an embodiment of the present invention.

FIG. 6 and FIG. 7 show schematic diagrams of optional display interfaces of the control method for the magnetic capsule endoscope control system according to an embodiment of the present invention.

### DETAILED DESCRIPTION

In order to enable those skilled in the art to better understand the application, the present invention can be described in detail below with reference to the accompanying drawings and preferred embodiments. However, the embodiments are not intended to limit the invention, and obviously, the described embodiments are only a part of the embodiments of the present invention, but not all of them. In the absence of conflict, the embodiments of the present invention and the features in the embodiments can be combined with each other. All other embodiments obtained by those having ordinary skill in the art without creative work based on the embodiments of the present invention are included in the scope of the present invention.

It should be noted that the terms, such as “first” and “second”, as described in the specification and claims of the present invention and the accompanying drawings are used only to distinguish similar objects but do not necessarily describe a particular sequence or order. It should be understood that the terms used in this way are interchangeable where appropriate, so that the embodiments of the present invention described herein can be performed in a sequence other than those diagrammed or described herein. In addition, the terms such as “comprise”, “include” and “have” and any of their variants are intended to cover non-exclusive inclusions. For example, for a process, method, system, product or device that comprises a series of steps or units, it is not necessarily limited to list all the steps or units, but other unlisted or inherent steps or units can also be included in such process, method, product or device.

### Embodiment 1

In this embodiment, a control method for a magnetic capsule endoscope control system is provided. The control method is applied to the magnetic capsule endoscope control system (Hereinafter referred to as “control system”). The control system comprises a controlled device, a local industrial personal computer (IPC) and a magnetic capsule endoscope. The local IPC is used to control the controlled device, and the controlled device comprises a magnetic moving part and a drive mechanism for controlling the movement of the magnetic moving part.

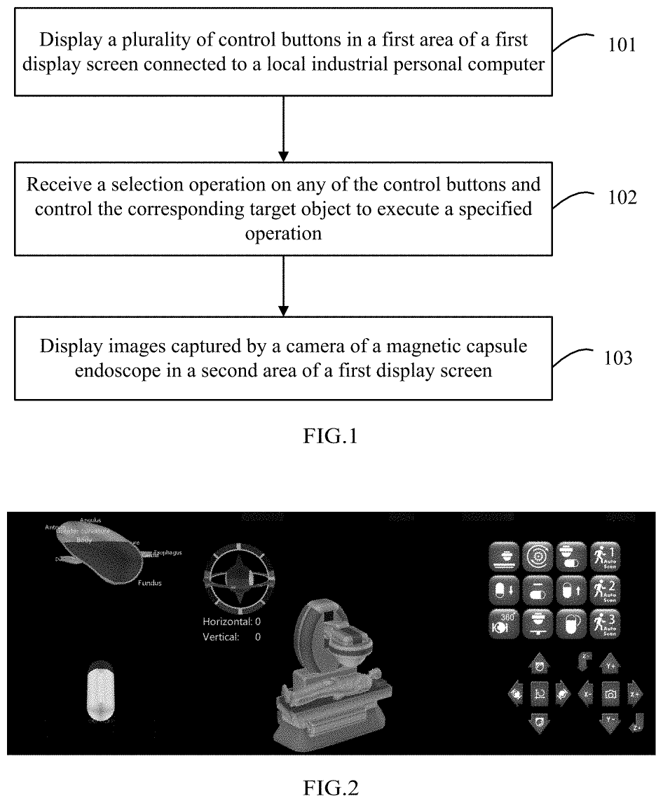

As shown in FIG. 1, the control method of the magnetic capsule endoscope control system provided in this embodiment comprises the following steps:

Step 101: displaying a plurality of control buttons in a first area of a first display screen connected to the local IPC, where each control button is used to issue a corresponding control instruction, and the control instruction is used to instruct a target object to execute a specified operation, and the target object can be the drive mechanism, the magnetic capsule endoscope or a camera of a control device;

Step 102: receiving a selection operation on any of the control buttons and control the corresponding target object to execute the specified operation;

Step 103: displaying images captured by a camera of the magnetic capsule endoscope in a second area of the first display screen.

Further, the method further comprises: displaying a schematic diagram of the current status of the drive mechanism in a third area of the first display screen; and/or displaying a schematic diagram of the current status of the magnetic capsule endoscope in a fourth area of the first display screen.

Referring to FIG. 2, showing a schematic diagram of an optional display interface of the control method for the magnetic capsule endoscope control system according to an embodiment of the present invention. In the display interface, the right area displays control buttons for a plurality of control instructions, the middle area displays the current status of the controlled device, including the human body and the deflection angle of the magnetic moving part (sphere) of the controlled device, the lower left area displays the current status of the magnetic capsule endoscope, and the upper left area displays the status of the entire stomach.

Optionally, the control system further comprises a remote control equipment connected to the local IPC through a telecommunication connection to control the controlled device. The local IPC is installed with a server, the remote control equipment is installed with a client corresponding to the server, and the controlled device also comprises a camera disposed towards an examination table of the controlled device. The remote control equipment communicates with the IPC through the telecommunication connection. The physical layer of the telecommunication connection can comprise fiber optical fiber communication, cable communication, and 3G, 4G and 5G wireless communication.

Furthermore, the method provided in this embodiment further comprises the following steps:

Step 11: establishing a telecommunication connection between the local IPC and the remote control equipment;

Step 12: displaying a plurality of control buttons in a first area of a second display screen connected to the remote control equipment;

Step 13: displaying the images captured by the camera of the controlled device in a second area of the second display screen.

Further, a schematic diagram of the current status of the drive mechanism can be displayed in a third area of the second display screen, and a schematic diagram of the current status of the magnetic capsule endoscope can be displayed in a fourth area of the second display screen.

In addition, the images captured by the camera of the magnetic capsule endoscope can be displayed in a fifth area of the second display screen.

Optionally, the control system further comprises a remote control equipment connected to the local IPC through a telecommunication connection to control the controlled device, and a console connected to the remote control equipment. The local IPC is installed with a server, the remote control equipment is installed with a client corresponding to the server, and the controlled device further comprises a camera disposed towards the examination table of the controlled device. The remote control equipment communicates with the IPC through the telecommunication connection. The physical layer of the telecommunication connection can comprise optical fiber communication, cable communication, and 3G, 4G and 5G wireless communication. The console and the remote control equipment can communicate through a wired communication interface, such as USB serial interface, and can also communicate through wireless communication. The console also comprises a touch panel. The touch panel can be a touch screen that displays one or more control buttons. The corresponding function of each button can be implemented once the button is touched.

Furthermore, the method provided in this embodiment further comprises the following steps:

Step 31: establishing a telecommunication connection between the local IPC and the remote control equipment and a connection between the remote control equipment and the console;

Step 32: displaying a plurality of control buttons on the touch screen of the console connected to the remote control equipment;

Step 33: displaying the images captured by the camera of the controlled device in a first area of a third display screen connected to the remote control equipment.

Further, a second area of the third display screen can also display a schematic diagram of the current status of the drive mechanism. A third area of the third display screen can also display a schematic diagram of the current status of the magnetic capsule endoscope.

In addition, the images captured by the camera of the magnetic capsule endoscope can also be displayed in a fourth area of the third display screen.

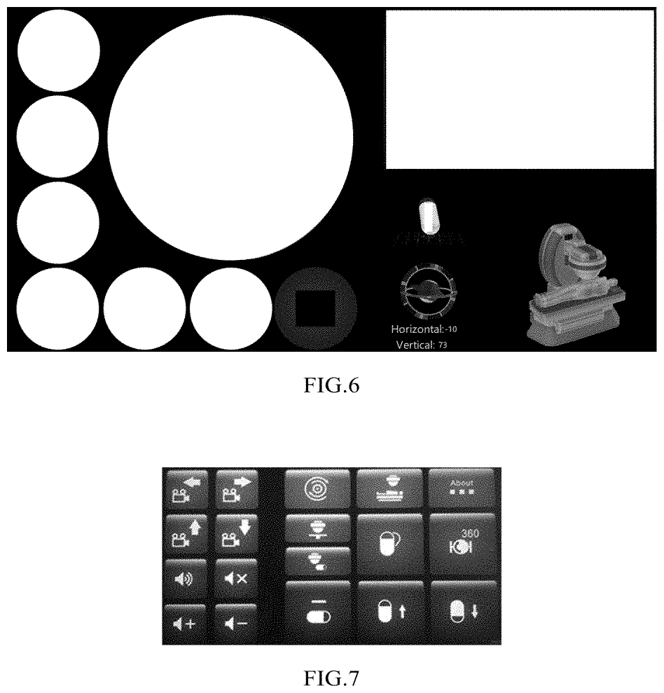

FIG. 6 and FIG. 7 show schematic diagrams of an optional display interface of the control method for the magnetic capsule endoscope control system according to an embodiment of the present invention. FIG. 7 shows the control buttons for a plurality of control instructions in the console connected to the remote control equipment. In FIG. 6, the upper right area is used to display the images captured by the camera of the controlled device, the lower right area is used to display the current status of the human body, the deflection angle of the magnetic moving part (sphere) of the controlled device, and the current status of the magnetic capsule endoscope, and the left area is used to display the images captured by the camera of the magnetic capsule endoscope. A big circular area in the middle of the left area is used to display the current image being captured, and the surrounding small circular areas around are used to display the stored previous images.

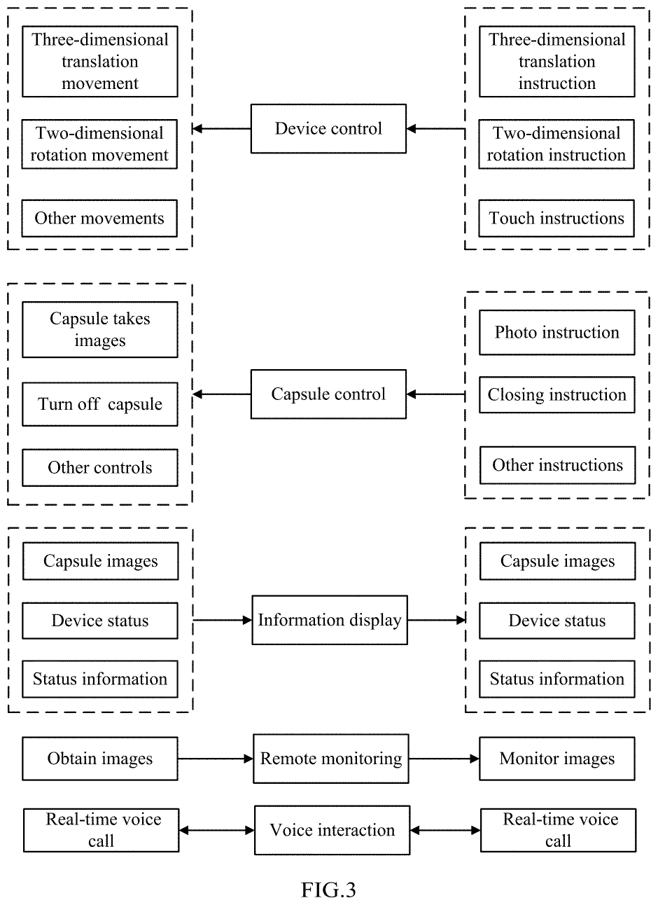

FIG. 3 shows a functional schematic diagram of the control method for the magnetic capsule endoscope control system provided in this embodiment.

The left side shows the functions implemented by the local IPC (through the server) of the controlled device, and the right side shows the functions implemented by the remote control equipment (through the client). Specifically, the functions can be categorized into device control, capsule control, information display, remote monitoring and voice interaction.

The device control function enables both the local IPC and the remote control equipment to implement three-dimensional translation movement, two-dimensional rotation movement, and other movements according to preset programs through control buttons.

The capsule control function enables both the local IPC and the remote control equipment to control the capsule endoscope through control buttons, such as controlling the capsule to take images, turning off the capsule or other capsule-related instructions.

The information display function displays the images taken by capsule, device status and other status information on the server in real time, and sends the relevant information to the client for display through the telecommunication connection. The movement status of the controlled device, including the real-time position and limit state of its three-axis translation, the angle of rotation, and the posture of the capsule, can be displayed in the local and remote interfaces in real-time animation to facilitate the inspection of the capsule endoscope.

The remote monitoring function can select a main control terminal between the client and the server, and the main control terminal can monitor the operation of the remote control equipment in real time and displays the images obtained by the server on the main control terminal in real time.

The voice interaction function can realize two-way real-time voice call and enable real-time communication by voice call between the client and the server.

In addition, an emergency stop instruction can be set to emergency stop the controlled device to ensure the safety of the remote operation. The network transmission of this instruction has the highest priority. The controlled device can be stopped after the instruction is issued by the client or the server.

Further, the plurality of control buttons can also comprise custom buttons for macro command editing. Specifically, an optional embodiment comprises the following steps:

Step 21: receiving editing operations to the custom buttons.

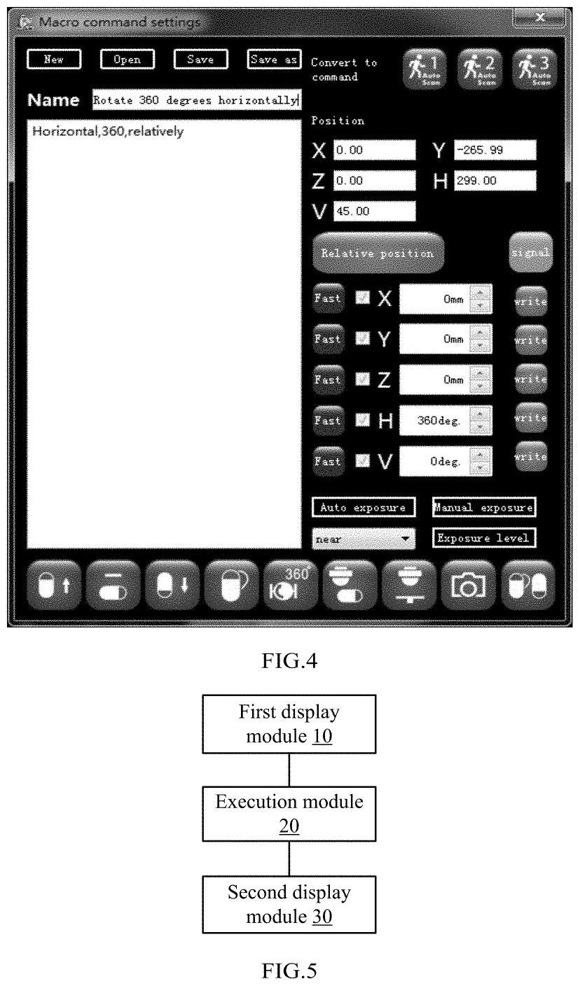

Step 22: displaying a macro command editing interface, wherein the macro command editing interface comprises one or more of the control buttons and a plurality of input boxes. The input boxes are used to receive parameter information of an input target position, and the parameter information comprises coordinate points and deflection angles. FIG. 4 shows an optional macro command editing interface, of which the right side is a plurality of input boxes that can be used to receive parameter information of the input target positions, and the bottom is one or more of the control buttons that represent a macro command.

Step 23: displaying the command currently edited in the macro command editing interface according to the control button selected and/or the parameter information of the target position input in the macro command editing interface, as shown in FIG. 4, wherein the command currently edited is displayed in the large input box in the left side of the interface.

Step 24: after determining to generate a macro command, generating a macro command from the command currently edited, and assigning the macro command to a custom button. After the macro command of the custom button is saved, it is executed once the custom button is selected.

It should be noted that the steps shown in the drawing can be performed on a computer system with a set of instructions that can be executed in a computer. Furthermore, although the logical sequence is shown in the flow chart, in some cases the steps shown or described here can be performed in a different sequence.

Through the above description of the embodiment, the person skilled in the art can clearly understand that the method in above embodiment can be implemented based on software and necessary general-purpose hardware platform, or only based on hardware, but in many cases the former is a better way. Based on this understanding, the technical solution of the present invention is essentially is implemented or the part that contributes to prior art is implemented in the form of a software product. The software product is stored in a storage medium (such as ROM/RAM, diskette, CD), and comprises a plurality of instructions to instruct a terminal device (a mobile phone, a computer, a server, or a network equipment, etc.) to execute the method described in each embodiment of the present invention.

### Embodiment 2

In this embodiment, a control device for the magnetic capsule endoscope control system is also provided. The control device is used to implement the above-mentioned embodiment 1 and its preferred embodiments. For terms or implementations not detailed in this embodiment, refer to the relevant descriptions in embodiment 1, and the described descriptions cannot be repeated.

The term “module” used hereafter refers to as a combination of software and/or hardware that can implement preset functions. Although the device described in the following embodiments is preferably implemented by software, the implementation by hardware or a combination of software and hardware can also be conceived.

The control device for the magnetic capsule endoscope control system provided in this embodiment is applied to the magnetic capsule endoscope control system. The control system comprises a controlled device, a local industrial personal computer (IPC) and a magnetic capsule endoscope. The local IPC is used to control the controlled device, and the controlled device comprises a magnetic moving part and a drive mechanism for controlling the movement of the magnetic moving part.

FIG. 5 shows a schematic diagram of the control device of the magnetic capsule endoscope control system according to an embodiment of the present invention. As shown in FIG. 5, the control device comprises a first display module 10, an execution module 20 and a second display module 30.

The first display module 10 is used to display a plurality of control buttons in the first area of the display screen connected to the local IPC, where each control button is used to issue a corresponding control instruction to instruct a target object to execute a specified operation, and the target object can be the drive mechanism, the magnetic capsule endoscope or the camera of the control device. The execution module 20 is used to receive a selection operation on any of the control buttons and to control the corresponding target object to execute the specified operation. The second display module 30 is used to display images captured by the camera of the magnetic capsule endoscope in the second area of the first display screen.

Further, the control device also comprises: a third display module for displaying a schematic diagram of the current status of the drive mechanism in the third area of the first display screen; and/or a fourth display module for displaying a schematic diagram of the current status of the magnetic capsule endoscope in the fourth area of the first display screen.

Further, the control system also comprises a remote control equipment connected to the local IPC through a telecommunication connection to control the controlled device. The local IPC is installed with a server, the remote control equipment is installed with a client corresponding to the server, and the controlled device also comprises a camera disposed towards the examination table of the controlled device. The device further comprises: a connecting module for establishing a telecommunication connection between the local IPC and the remote control equipment; a firth display module for displaying a plurality of control buttons in the first area of the second display screen connected to the remote control equipment; and a sixth display module for displaying images captured by the camera of the controlled device in the second area of the second display screen.

Further, the control system also comprises a remote control equipment connected to the local IPC through a telecommunication connection to control the controlled device and a console connected to the remote control equipment. The local IPC is installed with a server, the remote control equipment is installed with a client corresponding to the server, and the controlled device also comprises a camera disposed towards the examination table of the controlled device. The device further comprises:

an establishing module used to establish the telecommunication connection between the local IPC and the remote control equipment and a connection between the remote control equipment and the console; a twelfth display module used to display a plurality of control buttons in a touch screen of the console; a thirteenth display module used to display the images captured by the camera of the controlled device in the first area of the third display screen connected to the remote control equipment.

Further, the control device also comprises: a seventh display module used to display a schematic diagram of the current status of the drive mechanism in the third area of the second display screen; and/or an eighth display module used to display a schematic diagram of the current status of the magnetic capsule endoscope in the fourth area of the second display screen.

Further, the control device also comprises: a ninth display module used to display the images captured by the camera of the magnetic capsule endoscope in the fifth area of the second display screen. Further, the plurality of control buttons comprise custom buttons. The control device further comprises: a receiving module used to receive editing operations to the custom buttons; a tenth display module used to display a macro command editing interface that comprises one or more of the control buttons and a plurality of input boxes, the input boxes used to receive parameter information of the input target position, and the parameter information including coordinate points and deflection angles; a eleventh display module used to display the command currently edited in the macro command editing interface according to the control button selected in the macro command editing interface and/or the parameter information of the input target position; and a generating module used to generate a macro command from the command currently edited and to assign the macro command to the custom button after determining to generate the macro command, wherein the custom button executes the corresponding macro command after being selected.

It should be noted that each of the above modules can be implemented by software or hardware. In terms of implementation by hardware, the implementation method comprises, but not limited to: the above modules are all arranged in the same processor, or the above modules are arranged in different processors in the form of arbitrary combination.

Obviously, the person skilled in the art understand that the modules or steps of the present invention can be implemented with a common computing device and specifically, the modules or steps can be integrated on a single computing device or distribute on a network of a plurality of computing devices. Alternatively, the modules or steps can be implemented with the program code that can be executed on the computing device, so that they can be stored in the storage device for the computing device to execute. In some cases, the steps shown or described herein can be implemented in a different sequence. Or, they can be made into individual integrated circuit modules, or some of them can be made into a single integrated circuit module for implementation. In this way, the present invention is not limited to any specific combinations of hardware and software.

### Embodiment 3

In this embodiment, a storage medium is also provided. The storage medium stores computer programs and the computer programs are set to execute the steps in any of the above embodiments.

Alternatively, the above storage medium provided in the embodiment of the present invention may include, but not limited to, a medium that can store computer programs, such as a USB flash drive, a Read-Only Memory (ROM), a Random Access Memory (RAM), a mobile hard disk, a diskette, a CD, etc.

### Embodiment 4

In this embodiment, an electronic device is also provided, comprising a memory and a processor, where the memory stores computer programs and the processor is set to run the computer programs to execute the steps in any of the above embodiments.

Alternatively, the electronic device may also comprise a transmission device and an input and output device, wherein the transmission device is connected to the processor, and the input and output device is connected to the processor.

The person skilled in the art realizes that the present invention by no means is limited to the preferred embodiments described above. On the contrary, many modifications and variations are possible within the scope of the appended claims. Any modifications, equivalent substitutions, improvements, etc. made within the principles of the present invention are intended to be included within the scope of the present invention.

1. A control method for a magnetic capsule endoscope control system, characterized in that the control method is applied to the magnetic capsule endoscope control system, and the magnetic capsule endoscope control system comprises a controlled device, a local industrial personal computer and a magnetic capsule endoscope, the local industrial personal computer controls the controlled device, and the controlled device comprises a magnetic moving part and a drive mechanism for controlling the movement of the magnetic moving part;
wherein the control method comprises:
displaying a plurality of control buttons in a first area of a first display screen connected to the local industrial personal computer, wherein each of the control buttons issues a corresponding control instruction to instruct a target object to execute a specified operation, the target object is the drive mechanism, the magnetic capsule endoscope or a camera of a control device;
receiving a selection operation on any of the control buttons and controlling the corresponding target object to execute the specified operation;
displaying images captured by a camera of the magnetic capsule endoscope in a second area of the first display screen.

2. The method of claim 1, wherein the method further comprises:
displaying a schematic diagram of the current status of the drive mechanism in a third area of the first display screen; and/or,
displaying a schematic diagram of the current status of the magnetic capsule endoscope in a fourth area of the first display screen.

3. The method of claim 1, wherein the control system further comprises a remote control equipment connected to the local industrial personal computer through a telecommunication connection to control the controlled device, the local industrial personal computer is installed with a server, the remote control equipment is installed with a client corresponding to the server, and the controlled device comprises a camera disposed towards an examination table of the controlled device;
wherein the method further comprises:
establishing a telecommunication connection between the local industrial personal computer and the remote control equipment;
displaying a plurality of control buttons in a first area of a second display screen connected to the remote control equipment;
displaying images captured by the camera of the controlled device in a second area of the second display screen.

4. The method of claim 3, wherein the method further comprises:
displaying a schematic diagram of the current status of the drive mechanism in a third area of the second display screen; and/or,
displaying a schematic diagram of the current status of the magnetic capsule endoscope in a fourth area of the second display screen.

5. The method of claim 3, wherein the method further comprises:
displaying the images captured by the camera of the magnetic capsule endoscope in a fifth area of the second display screen.

6. The method of claim 1, wherein the control system further comprises a remote control equipment connected to the local industrial personal computer through a telecommunication connection to control the controlled device, and a console connected to the remote control equipment, the local industrial personal computer is installed with a server, the remote control equipment is installed with a client corresponding to the server, and the controlled device comprises a camera disposed towards an examination table of the controlled device;
wherein the method further comprises:
establishing a telecommunication connection between the local industrial personal computer and the remote control equipment and a connection between the remote control equipment and the console;
displaying a plurality of control buttons on a touch screen of the console connected to the remote control equipment;
displaying images captured by a camera of the controlled device in a first area of a third display screen connected to the remote control equipment.

7. The method of claim 1, wherein the plurality of control buttons comprise custom buttons;
wherein the method further comprises:
receiving editing operations to the custom buttons;
displaying a macro command editing interface, wherein the macro command editing interface comprises one or more of the control buttons and a plurality of input boxes, the input boxes receives parameter information of an input target position, and the parameter information comprises coordinate points and deflection angles;
displaying a command currently edited in the macro command editing interface according to the control button selected and/or the parameter information of the target position input in the macro command editing interface;
after determining to generate a macro command, generating a macro command from the command currently edited, and assigning the macro command to a custom button.

8. A control device for a magnetic capsule endoscope control system, characterized in that the control device is applied to the magnetic capsule endoscope control system, and the magnetic capsule endoscope control system comprises a controlled device, a local industrial personal computer and a magnetic capsule endoscope, the local industrial personal computer controls the controlled device, and the controlled device comprises a magnetic moving part and a drive mechanism for controlling the movement of the magnetic moving part;
wherein the control device comprises:
a first display module, for displaying a plurality of control buttons in a first area of a first display screen connected to the local industrial personal computer, wherein each of the control buttons issues a corresponding control instruction to instruct a target object to execute a specified operation, the target object is the drive mechanism, the magnetic capsule endoscope or a camera of the control device;
an execution module, for receiving a selection operation on any of the control buttons and controlling the corresponding target object to execute the specified operation;
a second display module, for displaying images captured by a camera of the magnetic capsule endoscope in a second area of the first display screen.

9. A storage medium, wherein the storage medium stores computer programs, the computer programs being set to execute a control method for a magnetic capsule endoscope control system, characterized in that the control method is applied to the magnetic capsule endoscope control system, and the magnetic capsule endoscope control system comprises a controlled device, a local industrial personal computer and a magnetic capsule endoscope, the local industrial personal computer controls the controlled device, and the controlled device comprises a magnetic moving part and a drive mechanism for controlling the movement of the magnetic moving part;
wherein the control method comprises:
displaying a plurality of control buttons in a first area of a first display screen connected to the local industrial personal computer, wherein each of the control buttons issues a corresponding control instruction to instruct a target object to execute a specified operation, the target object is the drive mechanism, the magnetic capsule endoscope or a camera of a control device;
receiving a selection operation on any of the control buttons and controlling the corresponding target object to execute the specified operation;
displaying images captured by a camera of the magnetic capsule endoscope in a second area of the first display screen.

10. (canceled)
